(19) United States
(12) Patent Application Publication
(10) Pub. No.: US 2024/0079083 A1
(43) Pub. Date: Mar. 7, 2024

(54) USING BIPARTITE NETWORKS TO DETERMINE INTERACTIONS BETWEEN ANALYTES AND CHEMICAL TREATMENTS

(71) Applicant: UT-Battelle, LLC, Oak Ridge, TN (US)

(72) Inventors: Muralikrishnan Gopalakrishnan Meena, Oak Ridge, TN (US); Matthew J. Lane, Oak Ridge, TN (US); Armin Guntram Geiger, Knoxville, TN (US); Daniel Allan Jacobson, Oak Ridge, TN (US); Joanna Tannous, Oak Ridge, TN (US); Tomas A. Rush, Oak Ridge, TN (US)

(21) Appl. No.: 18/243,320

(22) Filed: Sep. 7, 2023

Related U.S. Application Data
(60) Provisional application No. 63/404,197, filed on Sep. 7, 2022.

(51) Int. Cl. G16B 5/00; G16B 40/10

(52) U.S. Cl. CPC G16B 5/00; G16B 40/10

(57) ABSTRACT

A data-driven algorithm including various network analysis routes to characterize the production of known and putative specialized metabolites and unknown analytes triggered by different exogenous compounds. Bipartite networks quantify the relationship between metabolites and treatments stimulating their production through two routes. A direct route determines the production of known and putative specialized metabolites induced by a treatment. An auxiliary route is specific for unknown analytes. Various network centrality metrics rank treatments based on their ability to trigger a broad range of specialized metabolites. The specialized metabolites are ranked based on their receptivity to various treatments. This enables tracking the influence of any exogenous treatment or abiotic factor on metabolomics output for targeted metabolite research.

### BACKGROUND OF THE INVENTION

This invention was made with government support under Contract No. DE-AC05-00OR22725 awarded by the U.S. Department of Energy. The government has certain rights in the invention.

The present invention relates to metabolite prediction.

Potential of the diverse chemistries present in natural products for biotechnology and medicine remain largely untapped. One basic workflow for conventional metabolite prediction includes a data input block, analysis block, and post-analysis block. In the data input block, spectral data is produced or accessed. Next, the analysis block builds, then analyzes, molecular networks. For example, input mass spectrometry spectra can be converted into molecular networks by spectral alignment techniques and/or matching spectra to similar GNPS spectral libraries, using a variety of techniques (e.g., matching spectra based on cosine similarity). Then, in a post-analysis block, new molecules can be identified, and metabolite prediction can be done (e.g., clustering to identify matches in a database and identify new molecules). Visualization of spectral families and propagation of spectral annotation can also be performed.

This type of conventional solution for metabolite prediction has many shortcomings. For example, it does not have a quantifiable workflow to determine influence of chemical treatments on secondary metabolite elucidation. It does not have a quantifiable metric to rank the importance of the chemical treatments and the secondary metabolites. And, it has no way to track treatment effect to a metabolomics output. Further these types of conventional solutions are merely generic responses to a mass metabolomics database like the Global Natural Products Social Molecular Networking (GNPS) spectrometry database.

FIG. 6 shows a general prior art preprocessing algorithm 600 for obtaining untargeted peaks from High-Performance Liquid Chromatography-Mass Spectrometry (HPLC-MS) output. This conventional solution is described in Castillo, Sandra et al. “Algorithms and Tools for the Preprocessing of LC-MS Metabolomics Data.” Chemometrics and Intelligent Laboratory Systems 108.1 (2011): 23-32. Chemometrics and Intelligent Laboratory Systems. Web and Azzollini, Antonio et al. “Dynamics of Metabolite Induction in Fungal Co-Cultures by Metabolomics at Both Volatile and Non-Volatile Levels.” Frontiers in Microbiology 9.FEB (2018): n. pag. Frontiers in Microbiology. Web. This output is then either inspected (manually or visually) via a dimensionality reduction plot (e.g., Principal Component Analysis (PCA), t-Distributed Stochastic Neighbor Embedding (t-SNE), etc.). This conventional solution for identifying untargeted metabolomics has a number of drawbacks. For example, exploring data by hand or via visual inspection is unwieldy at best and, noticing trends among peaks between similar treatments on a matrix is difficult.

### SUMMARY OF THE INVENTION

It is useful to identify and characterize natural products not produced under conventional culture conditions, which can have potential antibacterial properties against pathogenic bacteria, act as bio-activators promoting growth of symbiotic bacteria or act as therapeutic agents for cancer treatments.

The disclosed technologies include various network analysis routes to characterize production of known and putative specialized metabolites and unknown analytes triggered by different exogenous compounds. Bipartite networks can quantify relationships between metabolites and treatments stimulating their production through two routes. A direct route determines the production of known and putative specialized metabolites induced by a treatment and an auxiliary route is specific for unknown analytes. The disclosed embodiments can track the influence of exogenous treatments or abiotic factor on metabolomics output for targeted metabolite development.

One aspect of the present disclosure is directed to memory encoding instructions that, when executed by data processing apparatus, cause the data processing apparatus to perform certain operations. These operations can include accessing information relating to effects of chemical treatments on analyte production, building, based on the accessed information, a bipartite network including chemical treatment nodes and analyte nodes. The bipartite network quantitatively represents the effects of chemical treatments to trigger production of analytes. The instructions can also include instructions for analyzing the bipartite network to identify dominant chemical treatments among the chemical treatments and identify secondary metabolites among the analytes and outputting the identified dominant chemical treatments and the identified secondary metabolites. The information relating to effects of chemical treatments on analyte production can include liquid chromatography mass-spectroscopy (LC-MS) spectra of the analytes corresponding to the chemical treatments.

In some embodiments, analyzing the bipartite network includes at least one of analyzing the bipartite network via a direct route to identify known and putative secondary metabolites and analyzing the bipartite network via an auxiliary route to identify untargeted and unknown analytes of interest.

With regard to the direct route approach, the analyte nodes of the built bipartite network are either known secondary metabolites or putative secondary metabolites or both, and the analyzing of the bipartite network analysis includes identifying the most influenced secondary metabolites from among the known or putative secondary metabolites.

The instructions for building the bipartite network can include defining two bipartite sets of nodes, one set including chemical treatments and the other including analytes. Further, the instructions can include instructions for constructing directional, weighted edges between nodes from each of the sets of nodes using log2fold change of an analyte by a chemical treatment and assigning positive or negative sign to each edge for representing metabolite upregulation or metabolite downregulation.

The instructions for analyzing can include instructions for computing a plurality of network centrality measures of the bipartite network including out-degrees for each chemical treatments, in-degrees for each analyte, broadcasting rank for each chemical treatment, and receiving rank for each analyte. The broadcasting ranks and receiving ranks can be normalized PageRank measures.

Another aspect of the present disclosure is generally directed toward memory encoding instructions that, when executed by a data processing apparatus, cause the data processing apparatus to perform operations including accessing spectra of unknown analytes relating to chemical treatments, generating a matrix relating the spectra of the unknown analytes to the chemical treatments, applying fold change rank order statistics (FCROS) to the matrix to determine a p-value and an f-value for each unknown analyte, building a bipartite network using unknown analytes with statistically significant p-values and f-values, selecting one or more unknown analytes by fold change or edge degree, and identifying secondary metabolites from among the selected one or more unknown analytes.

The instructions for applying fold change rank order statistics (FCROS) to the matrix can include repeatedly, for all combinations of controls and treatments: selecting a control sample and a treatment sample, computing a fold change for each analyte, and ranking analytes in increasing order to obtain an associated rank with each analyte. The instructions can further include computing an average of ranks for each analyte, using the mean and variance of the average of ranks to generate a normal distribution to associate a probability with each rank, and defining two cutoff values to identify up- and down-regulated analytes. An analyte can be classified as downregulated if below a first cutoff value and upregulated if above a second cutoff value.

The instructions for building the bipartite network can include repeatedly, for each treatment: selecting a treatment-specific FCROS matrix, in response to an analyte in the matrix having significant f-value and p-value, generating a treatment graph connecting all analyte nodes to a single node representing a treatment type associated with the treatment specific FCROS matrix, and representing edges between nodes and treatment type by fold change. Further, the instructions can include instructions for unioning the treatment graphs to generate a full union of all graphs and a network of similar treatments.

The instructions for selecting one or more unknown analytes by fold change or edge degree can include scoring the one or more analytes by at least one of degree connected to a singular treatment, upregulation value, downregulation value, and shared analytes between similar treatments.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components. Any reference to claim elements as “at least one of X, Y and Z” is meant to include any one of X, Y or Z individually, and any combination of X, Y and Z, for example, X, Y, Z; X, Y; X, Z; and Y, Z.

### BRIEF DESCRIPTION OF THE DRAWINGS

FIG. 1 illustrates a representative block diagram of exemplary methods of determining interactions between analytes and chemical treatments in accordance with the present disclosure.

FIG. 2 illustrates a representative block diagram of an exemplary method of determining interactions between analytes and chemical treatments with a direct route approach.

FIG. 3A illustrates an exemplary bipartite network for use in connection with the method of FIG. 2.

FIG. 3B illustrates a table of broadcasting values for various treatments of the bipartite network of FIG. 3A.

FIG. 3C illustrates table of receiving values for various analytes of the bipartite network of FIG. 3A.

FIG. 4 illustrates a representative block diagram of an exemplary method of determining interactions between analytes and chemical treatments with a direct route approach that emphasizes the step of building a bipartite network.

FIG. 5 illustrates a representative block diagram of an exemplary method of determining interactions between analytes and chemical treatments with a direct route approach that emphasizes the step of computing various network centrality measures.

FIG. 6 illustrates a prior art conventional method for processing raw spectra to identify known metabolites among corresponding analytes.

FIG. 7 illustrates a representative block diagram of an exemplary method of determining interactions between analytes and chemical treatments with an auxiliary route approach.

FIG. 8 illustrates an exemplary bipartite network for use in connection with the method of FIG. 7.

FIG. 9 illustrates a representative block diagram of an exemplary method of determining interactions between analytes and chemical treatments with an auxiliary route approach that emphasizes the step of applying fold change rank order statistics.

FIG. 10 illustrates a representative block diagram of an exemplary method of determining interactions between analytes and chemical treatments with an auxiliary route approach that emphasizes the step of building bipartite networks using analytes with statistically significant p-values and f-values.

FIG. 11 illustrates a representative block diagram of an exemplary method of determining interactions between analytes and chemical treatments with an auxiliary route approach that emphasizes the step of selecting high-scoring analytes.

FIGS. 12A-G illustrates an exemplary graph analysis characterizing influential species sections of Trichoderma for identifying putative compounds or metabolites.

FIGS. 13A-D illustrates network analysis-based direct and auxiliary routes for revealing the relationship between treatments and metabolite production in Aspergillus fumigatus at 25° C.

FIGS. 14A-D illustrates network analysis-based direct and auxiliary routes for revealing the relationship between treatments and metabolite production in Aspergillus fumigatus at 37° C.

FIG. 15 illustrates an exemplary bipartite network framework in accordance with the present disclosure.

### DESCRIPTION OF THE CURRENT EMBODIMENT

The present disclosure provides a bipartite network to determine interactions between analytes and chemical treatments. Network analysis methodologies, including direct and auxiliary approaches, provide suitable network analysis of analytes and chemical treatment interactions.

Triggering silent biosynthetic gene clusters (BCGs) in fungi to produce specialized metabolites is a generally tedious process that involves assessing various environmental conditions, applications of epigenetic modulating agents, or co-cultures with other microbes. The present disclosure provides data-driven solutions using bipartite network analysis. One system and method use a direct route to characterize the production of known and putative specialized metabolites triggered by various exogenous compounds. Another system and method use an auxiliary route to distinguish unique unknown analytes amongst the abundantly produced analytes in response to treatments. The systems and methods of this disclosure can assist researchers to identify treatments or applications that can positively influence the production of a targeted metabolite or recognize unique unknown analytes that can be further fractionated, characterized, and screened for their biological activities and hence, discover new metabolites.

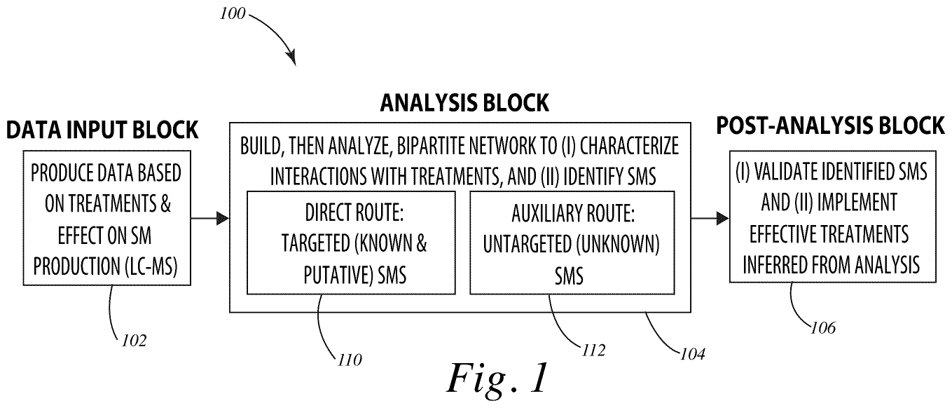

Referring to FIG. 1 the secondary metabolite and treatment ranking prediction method 100 includes a data input block 102, a data analysis block 104, and a post-analysis block 106. The data input block 102 produces data based on treatments and effect on secondary metabolite production. The analysis block 104, builds, then analyzes using a bipartite network to characterize interactions with treatments and identify secondary metabolites. This analysis block 104 includes analyzing one or more direct routes 110 and/or auxiliary routes 112 through the bipartite network. The direct route 110 is directed toward targeted (i.e., known and putative) secondary metabolites while the auxiliary route 112 is directed toward untargeted (i.e., unknown) secondary metabolites. The post-analysis block 106 validates identified secondary metabolites and generates effective treatments inferred from the analysis block 104.

In general, treatment refers to various conditions or interventions applied to a biological system, with the aim of inducing changes in the production, composition, or levels of secondary metabolites. Secondary metabolites refer to organic compounds that are produced by organisms, such as plants, fungi, and bacteria, but are not directly involved in the growth, development, or reproduction of the organism. Unlike primary metabolites, which are related to basic life functions, secondary metabolites often play a role in interactions with the environment, defense mechanisms, and communication with other organisms. Although the term secondary metabolite is utilized throughout this disclosure, however, it should be understood that the systems and methods of prediction and ranking can be utilized in connection with analytes generally and that the term secondary metabolite is being used interchangeably with analyte. That is, secondary metabolites can be considered analytes because an analyte generally refers to a substance under investigation.

In a bipartite network, the nodes are divided into two distinct sets, often referred to as partite sets.

The network analysis of the present disclosure provides a tool for accurately predicting the factors that can elucidate fungal metabolites and narrow down the list of BCGs to target. The network analysis of the present disclosure can facilitate targeting research to discover specialized metabolites (e.g., within Trichoderma) based on species-level taxonomic positioning and their predicted BCGs. It can also facilitate the discovery of new agents (e.g., Ilicicolin H, in Trichoderma reesei). The present disclosure emphasizes the assessment of the direct effect of exogenous treatments on the production of fungal specialized metabolites.

The systems and methods in accordance with the present disclosure facilitate tracking the influence of applied exogenous compounds on the production of characterized and putative metabolites as well as unknown analytes. Both approaches reveal treatments that dominate by triggering a variety of specialized metabolites. Moreover, unique specialized metabolites are also identified by these methods.

Targeted Metabolite Prediction and Treatment Ranking (Direct Route)

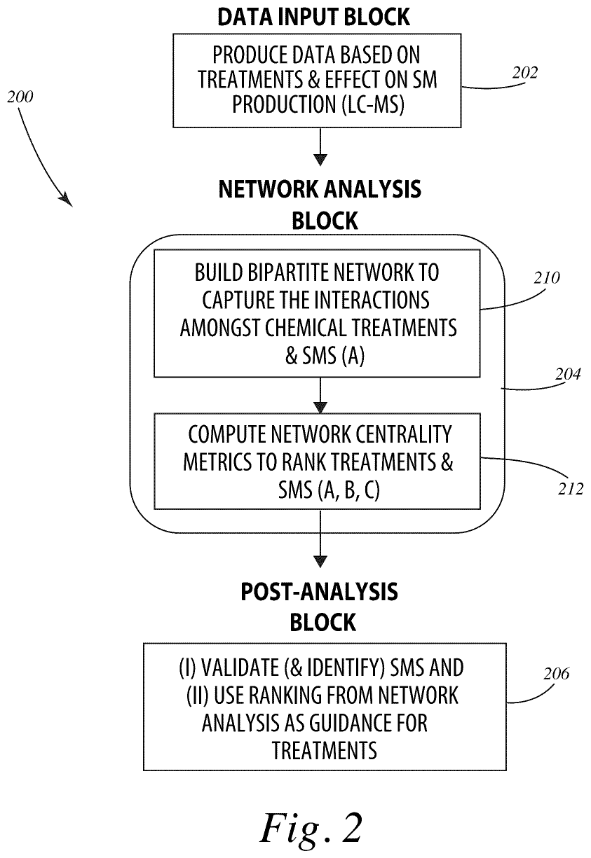

One aspect of the present disclosure is generally directed toward methods of secondary metabolite prediction and treatment ranking. Exemplary methodologies with a direct route implementation will now be described in more detail in connection with FIGS. 2-5.

In FIG. 2, an exemplary targeted metabolite prediction and treatment ranking method 200 includes a data input block 202 and post-analysis block 206 that can be the same or similar as described above in connection with FIG. 1. That is, the data input block 202 produces data based on treatments and effect on secondary metabolite production. The post-analysis block 206 validates identified secondary metabolites and generates effective treatments inferred from the analysis block 204.

The network analysis block 204 can be implemented using the direct route. In this embodiment, the direct route network analysis block 204 includes two steps 210, 212. First, the system builds a bipartite network to capture the interactions amongst chemical treatments and secondary metabolites 210. Then, the bipartite network is used to compute network centrality metrics to rank treatments and secondary metabolites 212.

The bipartite network framework represents interaction among chemical treatments and secondary metabolites. The bipartite network provides network centrality metrics. Network centrality metrics are quantitative measures for accessing the importance, influence, and prominence of nodes (individual entities) within a network. These metrics help to identify the most central or influential nodes in a network based on their connectivity patterns. In the current disclosure, the network centrality metrics can be utilized to rank effectiveness of treatments to influence secondary metabolites based on interactions on various secondary metabolites and receptivity of secondary metabolites to treatments based on overall effects from various treatments.

The bipartite network provides upregulation and downregulation of secondary metabolites by treatments visualized by directional, signed edges of the bipartite network. Upregulation and downregulation are terms used to describe changes in the expression or activity in a biological system. These changes can occur in response to various stimuli, such as environmental cues, developmental stages, diseases, or treatments. The bipartite network is useful in connection with models using up- or down-regulation of secondary metabolites by treatments, visualized by directional, signed edges of the network. For example, it can facilitate validation and confirmation (or identification) of known or putative metabolites through a targeted approach.

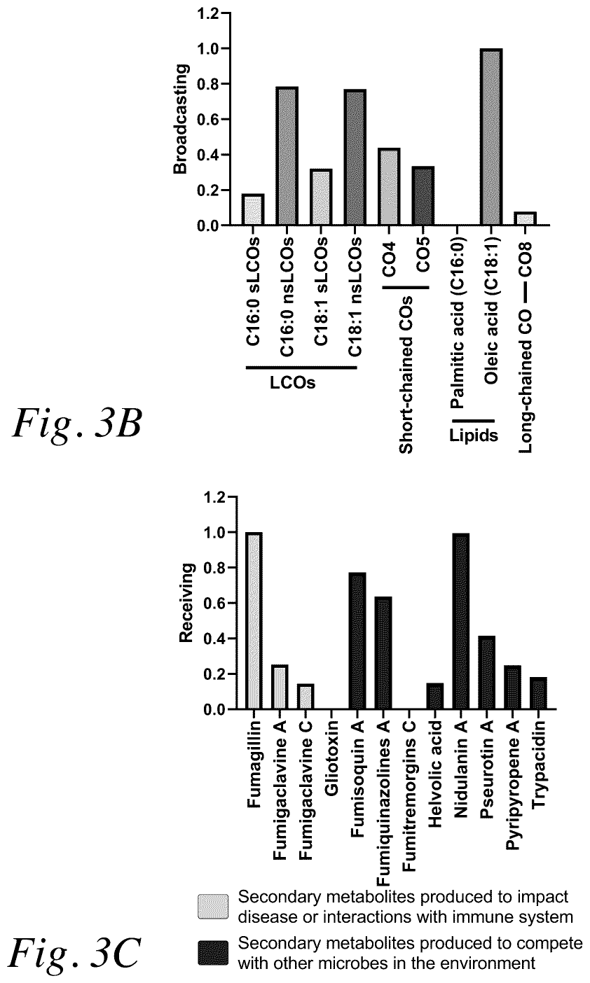

FIG. 4 illustrates a method for building a bipartite network to capture interactions among treatments and secondary metabolites 400. This embodiment emphasizes computing network centrality metrics to rank treatments and secondary metabolites based on direct routes through the bipartite network. The process includes a data-input block 402 that includes obtaining log2fold change of secondary metabolite concentration for each treatment, a network analysis block 403, and a post-analysis block 414. The network analysis block 403 includes building a bipartite network 401, which includes defining two types of nodes, treatment nodes 404 and secondary metabolite nodes 406. Next, directional, weighted edges between nodes are constructed 408 using the log2fold change of a secondary metabolite by a treatment. That is, the edges of the network are the log2fold change by a treatment on a particular secondary metabolite, which correlates with the biological relevance of how data is produced. The edges are directional (e.g., edges originate from a treatment toward a secondary metabolite) and are weighted to represent the change in production of that secondary metabolite because of that treatment (e.g., log2fold change). The bipartite network can facilitate visualizing metabolite regulation by assigning + or − signs (or some other corresponding visualization) to edges 410. Upregulation can be shown with a + sign or a red colored edge. Downregulation can be shown with a − sign or a blue colored edge. Arranging the bipartite network in this manner allows tracking how a specific treatment influences a metabolomic output through quantification and regulation. With the bipartite network built, network centrality metrics can be computed to rank treatments and secondary metabolites based on direct routes 412.

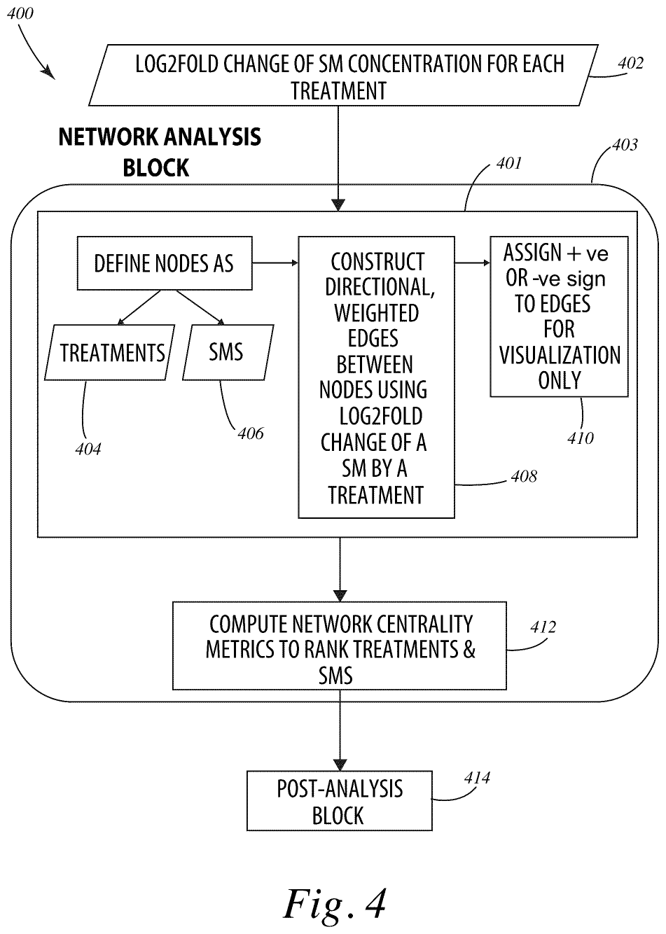

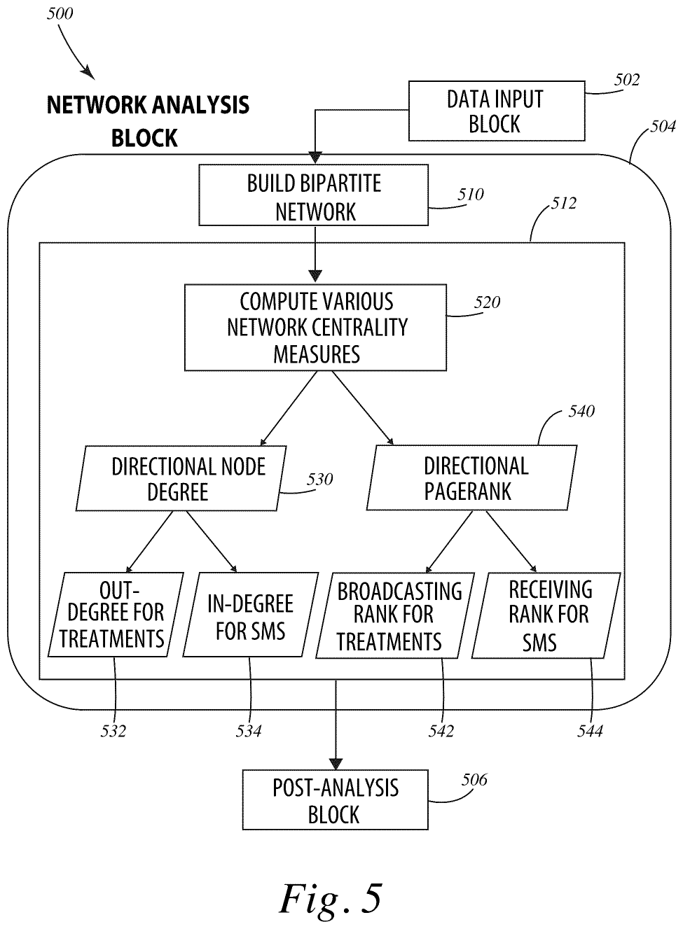

Another exemplary method of secondary metabolite prediction and treatment ranking with a direct route implementation 500 will now be described in more detail in connection with FIG. 5. FIG. 5 illustrates in more detail how network centrality metrics to rank treatments and secondary metabolites can be obtained and utilized. The data input block 502 is a step of the method that includes producing (e.g., collecting/accessing/obtaining from a user or data source) data based on treatments and effect on secondary metabolite production (e.g., treatments and secondary metabolites and their relationships). The analysis block 504 includes two separate parts, building a bipartite network 510 and computing centrality metrics 512. The analysis block 504 facilitates characterization of interactions with treatments and secondary metabolites. Exemplary construction of the bipartite network is discussed above in connection with FIG. 4. Exemplary methodologies for centrality metric computation will now be discussed in detail.

Centrality metric computation 512 can provide quantification of importance of treatments and secondary metabolites, validation and confirmation regarding putative metabolites, and identification of treatments that have the strongest or weakest influence on a specific metabolite of interest. In the illustrated embodiment, computing various network centrality metrics 520 includes measuring directional node degree 530 and measuring directional PageRank 540.

Node degree, a measure readily used in network science literature, gives total influence of treatments on secondary metabolites and receptivity of secondary metabolites to treatments. In this embodiment, an out-degree measurement 532 for treatments and an in-degree measurement 534 for secondary metabolites can be obtained. Ranking can be conducted based on known grouping of treatments and secondary metabolites (e.g., based on the out-degree and/or in-degree measurements). Out-strength generally refers to the number of connections or edges originating from nodes in one partite set and connecting to nodes in the other partite set. In other words, it represents the total degree of outgoing connections from nodes in a specific partite set to nodes in the other partite set. In-strength generally refers to the number of connections or edges directed towards nodes in a particular partite set from nodes in the other partite set. It represents the total degree of incoming connections to nodes in a specific partite set from nodes in the opposite partite set.

Edge weight generally refers to a numerical value assigned to a connection between nodes. This weight can represent a characteristic, significance, strength, or measure associated with the connection. In the current embodiment, the edge weights represent interaction/influence between chemical treatment nodes (top) and secondary metabolite nodes (bottom).

PageRank refers to the underlying method used by the Google search engine to rank a web page (e.g., as explained in the paper entitled “The Anatomy of a Large-scale Hypertextual Web Search Engine” by Sergey Brin and Lawrence Page, dated April 1998 available in the Computer Networks and ISDN Systems Journal). PageRank utilizes relative importance of nodes based on the relevance of its neighbors (e.g., who is connected to whom) to provide more specialized ranking of treatments and secondary metabolites. The method can obtain broadcasting rankings 542 for the treatment nodes based on which treatment nodes are most influential and obtain receiving rankings 544 for the secondary metabolite nodes based on which secondary metabolite nodes are most influenced nodes. These values can be min-max normalized (between 0-1). Utilizing the directional versions of these measures enables separate ranking of treatments and secondary metabolites, further emphasizing the relevance of generating a directional network.

These rankings (e.g., treatment out-degree 532, secondary metabolite in-degree 534, treatment broadcasting value 542, and secondary metabolite receiving value 544) can provide valuable insights. In the post-analysis block 506, these rankings can be (i) validated using chemical analysis of the known metabolites based on network analysis, (ii) potentially, use genetic knockouts to characterize gene clusters of putative metabolites, and (iii) used as a basis to recommend usage of treatments.

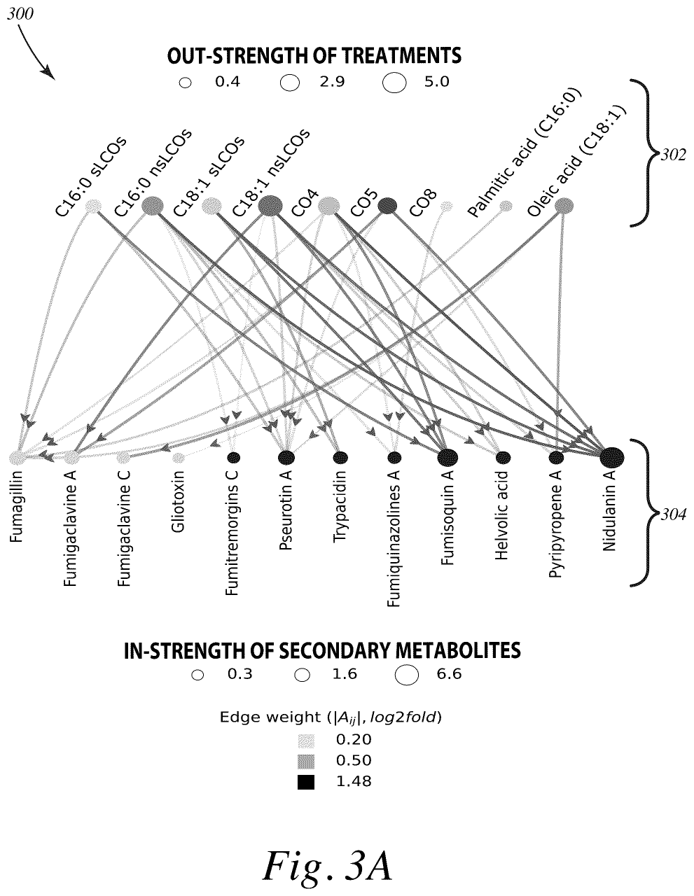

An exemplary bipartite network is illustrated in FIG. 3A and designated 300. One partite set of nodes 302 is provided along the top of the network visualization and includes various treatments with their out-strengths being depicted by the size of the node and the provided key. Another partite set of nodes 304 is provided along the bottom of the network visualization of FIG. 3A that includes various secondary metabolites along with their in-strength values being depicted by the size of the node and the provided key. The edge weights between the two partite sets are depicted with the darkness of the edge corresponding to a range of edge weights according to the provided key (e.g., log2fold change). The color of the edge represents whether the interaction is upregulation (red) or downregulation (blue).

FIG. 3B illustrates the treatment nodes from the treatment partite set of the bipartite network of FIG. 3A and their broadcasting values while FIG. 3C illustrates the secondary metabolite nodes from the secondary metabolite partite set of the bipartite network of FIG. 3A and their receiving values.

The bipartite network visualizations (e.g., as depicted in FIGS. 3A-C) also provide simple and effective visual representations of influence of treatments on secondary metabolites and receptivity of secondary metabolites from treatments.

The bipartite network 300 provides a quantifiable framework to represent interaction/influence between chemical treatments & secondary metabolites—using log2fold change from experiments. It also provides a quantifiable framework to rank treatments (based on effectiveness to influence secondary metabolites) and secondary metabolites (based on receptivity to treatments)—node degree and PageRank measures.

Identifying Untargeted Metabolomics (Auxiliary Route)

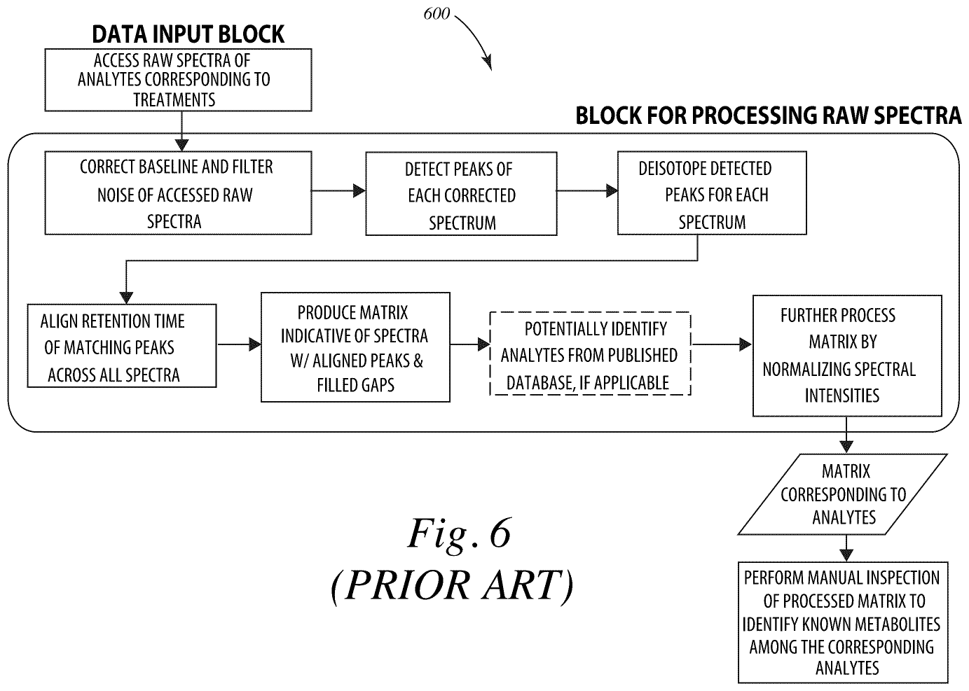

Another aspect of the disclosure involves identifying untargeted metabolomics, for example as illustrated in the method 700 of FIG. 7. This method 700 includes a data input block 702 that obtains access to raw spectra of analytes corresponding to treatments 710, which can be processed 712 to obtain a matrix corresponding to analytes 714. The network analysis block 704 then can apply fold change rank order statistics (FCROS) to the matrix to determine (p,f) values corresponding to analytes 720. Statistically significant (p, f) values can then be used to build a bipartite network 722. And high-scoring analytes can be selected by fold change or edge degree 724. In a post-analysis block 706, secondary metabolites from among the selected analytes can be identified.

This aspect provides application of FCROS toward Untargeted Analyte Peaks (e.g., traditionally used in Proteomic Analysis post LC-MS). In addition, this methodology enables generation of bipartite networks between application nodes and highly associated (determined via FCROS output) analyte peaks.

One of the benefits of this methodology is the quantification and regulation of unknown analytes through strict curation of peak data using conservative thresholds. By using conservative thresholds in the peak picking processing steps, confidence is achieved that the analytes associated with each peak are truly extant. Further, this methodology of identifying untargeted metabolomics provides specific tracking of which treatments correspond in analyte output. For example, analyte nodes connected with an edge to more than one treatment illustrate that both treatments statistically produce the same analyte obtained from the output sample.

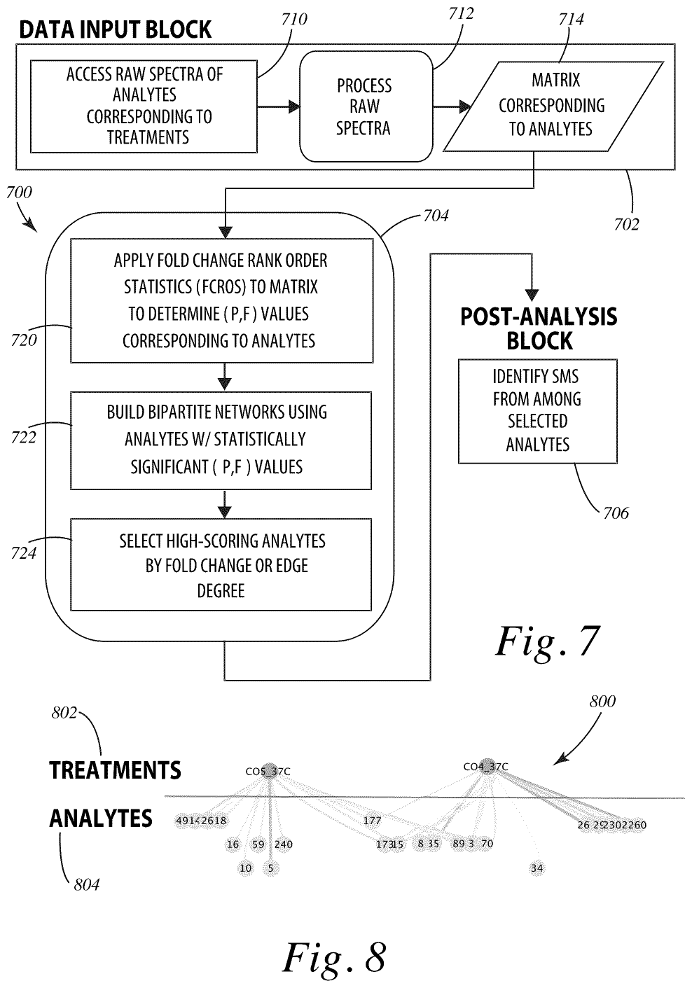

FIG. 8 illustrates an exemplary bipartite network 800 with two treatments 802 and several unknown analytes 804, identified by previously defined numerical identifiers. The color of the edge represents whether the interaction is upregulation (red) or downregulation (blue).

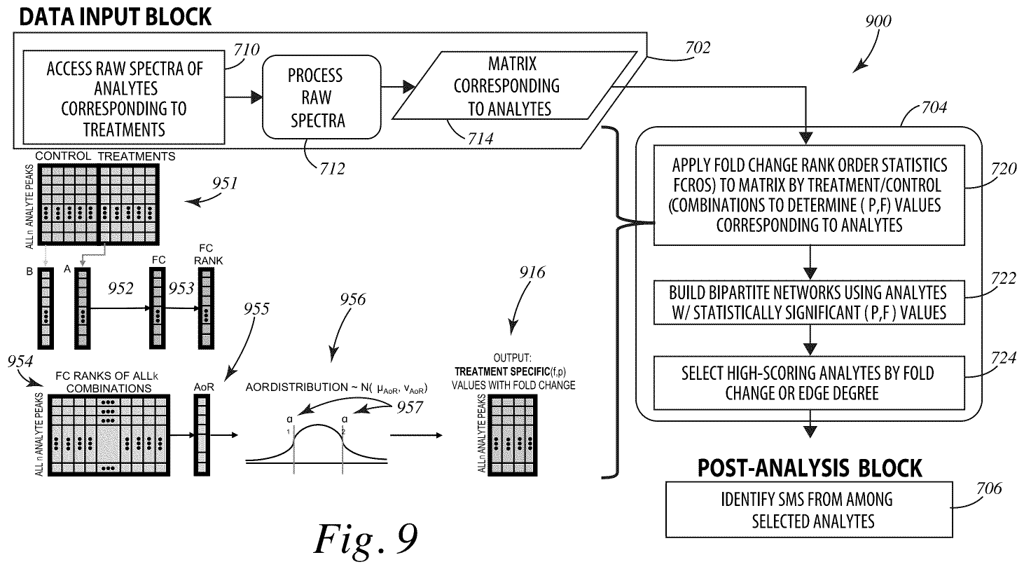

FIG. 9 illustrates an enhanced flowchart 900 showing a method of identifying untargeted metabolomics that emphasizes the application of fold change rank order statistics. This method 900 includes a data input block 702 that obtains access to raw spectra of analytes corresponding to treatments 710, which can be processed 712 to obtain a matrix corresponding to analytes 714. The network analysis block 704 then can apply fold change rank order statistics (FCROS) to the matrix to determine (p,f) values corresponding to analytes 720. A discussion of FCROS can be found at Dembélé, Doulaye, and Philippe Kastner. “Fold Change Rank Ordering Statistics: A New Method for Detecting Differentially Expressed Genes.” BMC Bioinformatics 15.1 (2014): n. pag. BMC Bioinformatics. Web.

The process of applying FCROS to the matrix 720 can include the following sub-steps:
Step 951—Select 2 samples: 1 Control, 1 Treatment
Step 952—Compute Fold Change for each analyte (1 . . . n)
Step 953 —Rank analytes in increasing order to obtain an associated rank with each analyte
Step 954—Repeat steps 1-3 for all k combinations of controls and treatments
Step 955—Compute the Average of Ranks (AoR) for each analyte
Step 956—Use the mean and variance of the AoR to generate a Normal Distribution to associate a probability with each rank
Step 957—Use two cutoff values a₁ and a₂ to identify up/down regulated analytes (downregulated if below a₁, upregulated if above a₂)

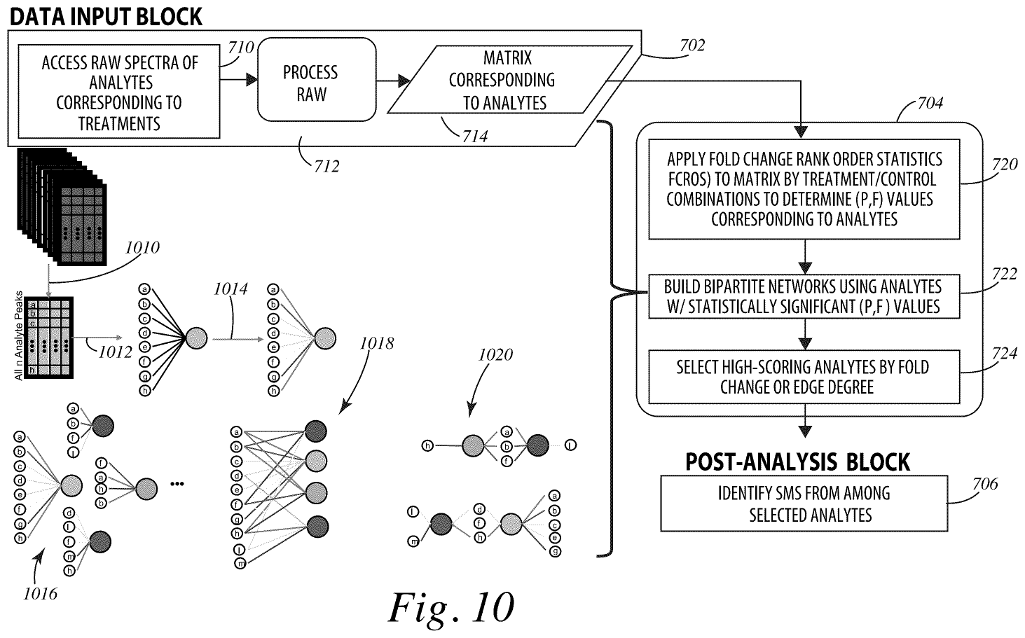

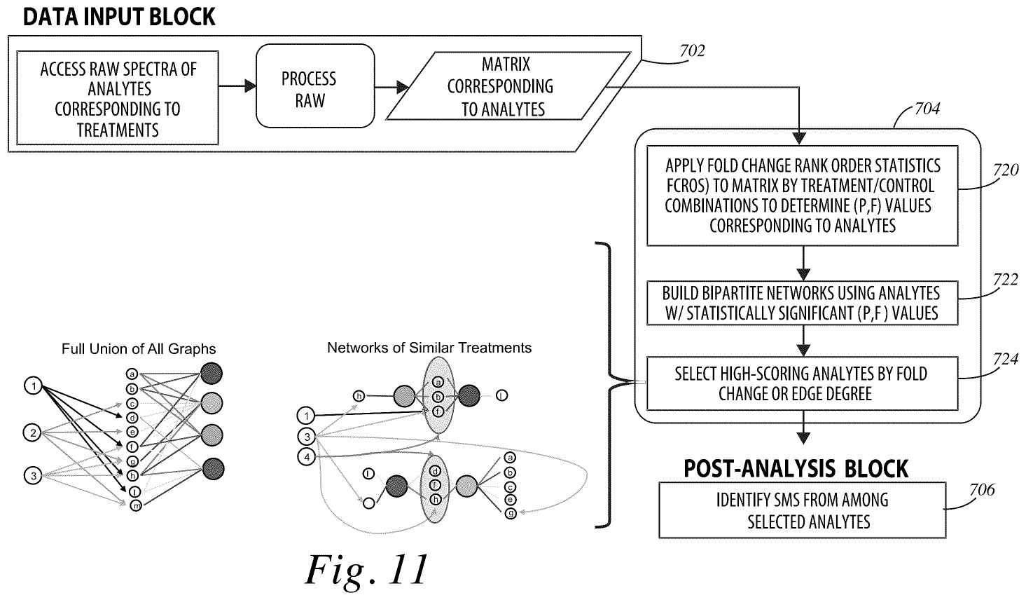

The output of this application of FCROS to the matrix 720 provides treatment specific (f, p) values with fold change 916. Statistically significant (p, f) values can then be used to build a bipartite network 722 and select high-scoring analytes 724, for example as explained in connection with FIGS. 10 and 11 respectively.

FIG. 10 shows a process that emphasizes building bipartite networks 722 with analytes with statistically significant (p, f) values. The following sub-steps can include:
Step 1010—Select a single treatment specific FCROS Output matrix;
Step 1012—If analyte (row) in matrix has significant (f,p) value, treat it as a node and connect all nodes to a single node labelled as the treatment type;
Step 1014—Colorize edges between nodes and treatment type by change (gradient from negative to positive)—the current embodiment utilizes log2 fold change;
Step 1016—Repeat steps 1010 to 1014 for each treatment;
Union treatment graphs including
   Step 1018—Conduct a full union of all graphs; and
   Step 1020—Generate networks of similar treatments.

High-scoring analytes can be selected by fold change or edge degree 724, for example as discussed in more detail in FIG. 11.

FIG. 11 shows a process that emphasizes selecting high-scoring analytes 724. This selecting step can include methods of scoring analytes favoring:
Analytes with both up and down regulation;
Analytes with degree 1 connected to a singular treatment (Note: searching for degree 1 nodes on sub networks of similar treatments will yield more analytes, e.g., nodes a, b, c, and e in sub network B have degree 1, however a, b, c, and e do not have degree 1 in the full union network);
Analytes with strong up or down regulation;
Shared analytes between similar treatments.

In a post-analysis block 706, secondary metabolites from among the selected analytes can be identified.

### Applications and Examples of the Bipartite Network Framework of Treatments and Metabolomic Outputs

Several examples of a bipartite network framework of treatments and metabolomic outputs will now be described. Bipartite networks are built to quantify the relationship between metabolites and the sources triggering their production, such as various exogenous biomolecules or compounds. As discussed above, a bipartite network (or graph) is a collection of nodes connected by lines named edges. The nodes represent the entities or elements of a system, and the edges represent the interaction or relationship amongst the features. For example, in cell metabolism, a metabolic network represents the biochemical reactions amongst substrates that result in products. The nodes of the metabolic network represent the substrates, and the edges represent the metabolic reactions amongst the substrates. For example, this framework can assess the effect of exogenous compounds on the production of specialized microbial metabolites. This relationship between treatments and specialized metabolites can be represented by a network, as shown in FIGS. 3A, 12A, 13A, and 14A. The nodes can be classified into two types: the treatments and the specialized metabolites, resulting in a bipartite network. The edges represent the magnitude of up- or down-regulation of specialized metabolites caused by the treatments compared to a controlled case (measured by the magnitude of log2 fold change using processed spectral data from targeted LC-MS analysis).

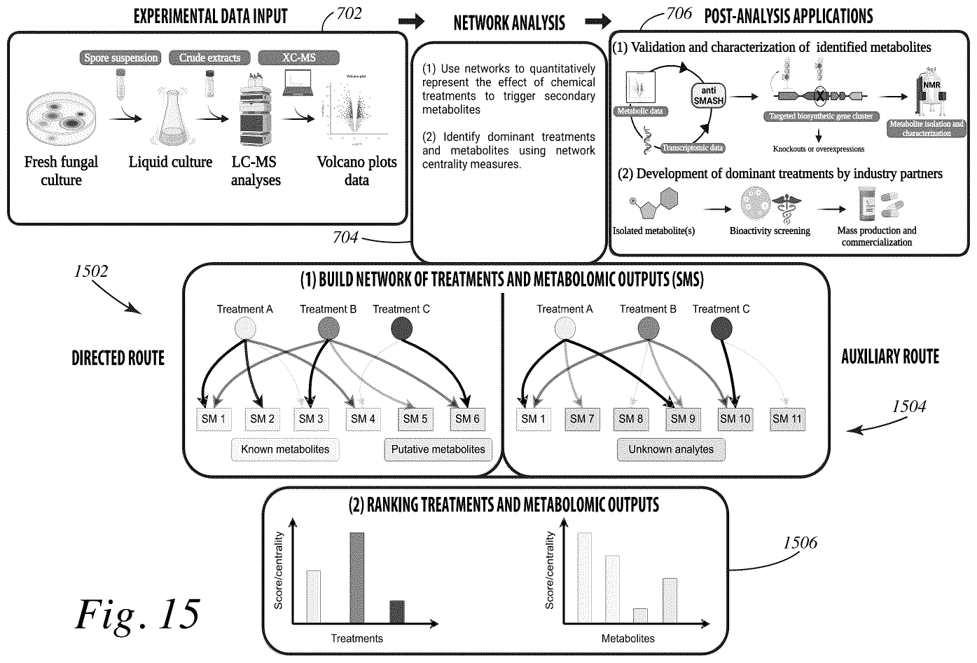

The bipartite network provides an in-depth quantification and clear visual representation of a treatment's ability to trigger the production of various specialized metabolites. Two routes are provided to assess specialized metabolite production using the bipartite network formulation, as shown in FIG. 15. The first is the direct route 1502 to determine the biosynthesis of known and putative metabolites, whereas the second is the auxiliary route 1504 to assess the production of unknown analytes. In the direct route 1502, the network nodes include treatments that elucidate known and putative metabolites by a microbe. Network centrality measurements 1506 are used to rank the treatments and the specialized metabolites. Those measurements can be used to identify the most influential nodes in the bipartite network. That is, the centrality measurements of node strength and PageRank can be used to identify the most effective treatments and metabolites. The treatments can be ranked based on their capability to trigger metabolite production, and the metabolites can be classified based on their popularity in being activated by various treatments. In the auxiliary route, the bipartite networks are built using analyte peaks extracted from post-processed spectral data. Further, the systems and methods of the present disclosure analyze the edges and neighborhoods of nodes to distinguish unique analytes among the total pool.

### Trichoderma Example

The discovery and usage of biological controls as management strategies started with astute observations of ecological niches for studying microbial interactions. Not all microbes associated with crops are harmful. Beneficial microbes have become a component of pest management strategies to control pest populations or promote plant health. Factors that influence the use of beneficial microbes as biological control products are stress-induced environments, nutrient-deficient areas, and known populations of plant pathogens that can be controlled. In general, the use of biological control products is preferred for many reasons, including the reduction of pesticide use, cost-effectiveness, and its efficacy against a broad range of natural pest and support services. Yet, biological control product applications face several challenges including invasive species stemming from the fungus used as an active ingredient; increasing crop groups, cultivars and varieties; pest complexes and resistances; incompatibility with pesticides; non-targeted effects, and risk assessment strategies.

Given the complexity of these challenges, this example discusses bioprospecting microbes, using Trichoderma species as model organisms. This example integrates predictive biology, functional genomics, high-throughput analytics, and next-generation biodesign and genome engineering approaches. Using this framework, predictions of which species among the already sequenced Trichoderma have unique potential as valuable biocontrol agents or source of natural products can be made.

The effects of Trichoderma species on other organisms are largely influenced by the production and secretion of metabolites, which have various established roles. Fungal metabolites have been reported to act either as communication signaling molecules between microorganisms and their hosts, or as defense agents in interactions with neighboring organisms. They were also shown to influence the development of the producing organism and to stimulate or inhibit the biosynthesis of other metabolites. Genes responsible for the biosynthesis of secondary metabolites are often arranged into clusters. Those clusters are regulated by environmental signals and by transcriptional and epigenetics modulators. Different classes of secondary metabolites reported in fungi are indole alkaloids, nonribosomal peptides (NRPs), polyketides, shikimic acid-derived compounds, and terpenoids. Although Trichoderma is one of the mass producers of secondary metabolites with 23 identified families, classes, or compounds, and some with genetic accessibility, little is known about the biosynthetic gene clusters responsible to produce those metabolites. Moreover, the level of diversity among secondary metabolites produced across known Trichoderma species is still largely indefinite.

Besides secondary metabolites, antimicrobial peptides (AMPs) are another resource for biological products. AMPs, a cell defense mechanism produced by many organisms, are short and generally positively charged peptides that can directly kill microbial pathogens by modulating the host defense system. There has been increased AMP research over the years because of concerns regarding the advent of a “post-antibiotic era”. In addition, bacterial resistance to AMPs has been shown to be low or potentially negligible. To date, there are more than 3,000 characterized AMPs based on their source, activity, structural characteristics, and amino acid composition. Many AMPs interact with membranes, causing cell wall inhibition and nucleic acid binding. Among other types, Trichoderma has a unique class of AMPs called peptaibols that include rare amino acids in their sequences, which provide resistance to the host or pathogen proteases and induce programmed cell death in plant fungal pathogens. Recent technological and computational advancements are expected to improve their classification, exploration, and characterization.

It can be as much as 10 years before a newly discovered biological control agent is released. Therefore, a system and method for guiding researchers thorough an experimental plan to discover a novel product and implement it into the market can be helpful. Two starting points, an omics road or biodesign road, can predict and identify putative natural products. Using reference genomes, the omics road queries candidate species for predicted backbone enzymes, putative metabolites, or annotated proteins relevant to biocontrol. Given the dynamic nature of genome expression, computational approaches (like machine-learning or graph theoretical methods) benefit greatly from the addition of functional genomics data, e.g., transcriptomics, proteomics, and metabolomics. In parallel or separately, the challenges of linking predictable gene clusters to their corresponding compounds can be addressed by following the biodesign road to extract putative metabolites, isolate them, and test for bioactivity. Both roads merge at the implementation step, where the metabolite characterized for specific bioactivity can be used as a biological control product. The implementation can determine the compound, its bioactivity, the gene(s) or biosynthetic pathways responsible for its production, and its potential use in a greenhouse or field setting. Collectively, this system and methodology provides insightful experimental planning that can allow for faster approval of a novel biological control product into the market.

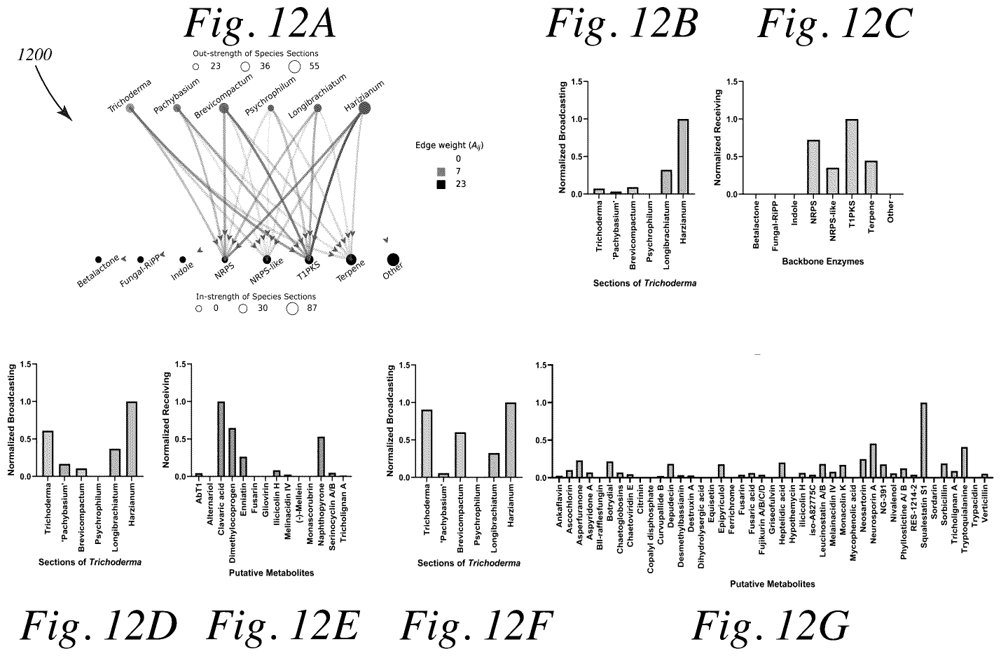

To quantify the uniqueness and predictive diversity of natural products across the Trichoderma sections or functional categories of secondary metabolites, a bipartite network 1200 (See FIG. 12A) can be used to represent the relationships between or influence of results from bioinformatics tools (e.g., Antibiotics & Secondary Metabolite Analysis Shell) across different Trichoderma sections. The bipartite network (or graph) includes nodes and edges and, in this context, the sections and backbone enzymes represent the nodes, and the edges are weighted by the average number of times a category is observed by all the species in a section.

Two different measures can be used to quantify and rank the importance of sections and backbone enzymes. These are (1) the strength and (2) PageRank of the nodes. The strength of the node is determined by the summation of all the edges from (out-strength) or to (in-strength) a node. The PageRank measure quantifies the relative importance of a node (section or backbone enzyme) based on the connections it has. The sections and the enzymes are ranked for being the most influential and influenced nodes, respectively, using the directed PageRank measures broadcasting and receiving measures, as shown in FIGS. 12B-C. These values are min-max normalized (between 0-1). The above network analysis can be performed separately for the interaction among the sections and putative metabolites with >75% and <75% matched sequences with known metabolites. The min-max normalized broadcasting and receiving PageRank values for these analyses can be reported by the system, as shown in FIGS. 12D-G.

Aspergillus Example @ 25° C.

The bipartite network framework was used in this example to reveal the effect of various chitooligosaccharides and lipid treatments on triggering the production of specialized metabolites in Aspergillus fumigatus. Various chitooligosaccharides and lipids were applied as exogenous treatments since they are common constituents found in most fungi. Moreover, it has been previously shown that lipids influence fungal metabolomics; however, the impacts of chitooligosaccharides have remained unknown. In contrast, chitooligosaccharides are reported to have antifungal activity, which might potentially influence the metabolomic profile in Aspergillus species. This example highlights the influence of temperature on the production of specialized metabolites by conducting the experiments at 25 and 37° C. Aspergillus fumigatus is generally examined at 25° C. to explore the extent of its metabolomic capabilities or its lifestyle as a soilborne saprotroph that recycles environmental carbon and nitrogen. However, the fungus is also an opportunistic human pathogen and is commonly examined at 37° C. for its ability to cause aspergillosis, a lung disease found in immunocompromised patients.

Direct Route

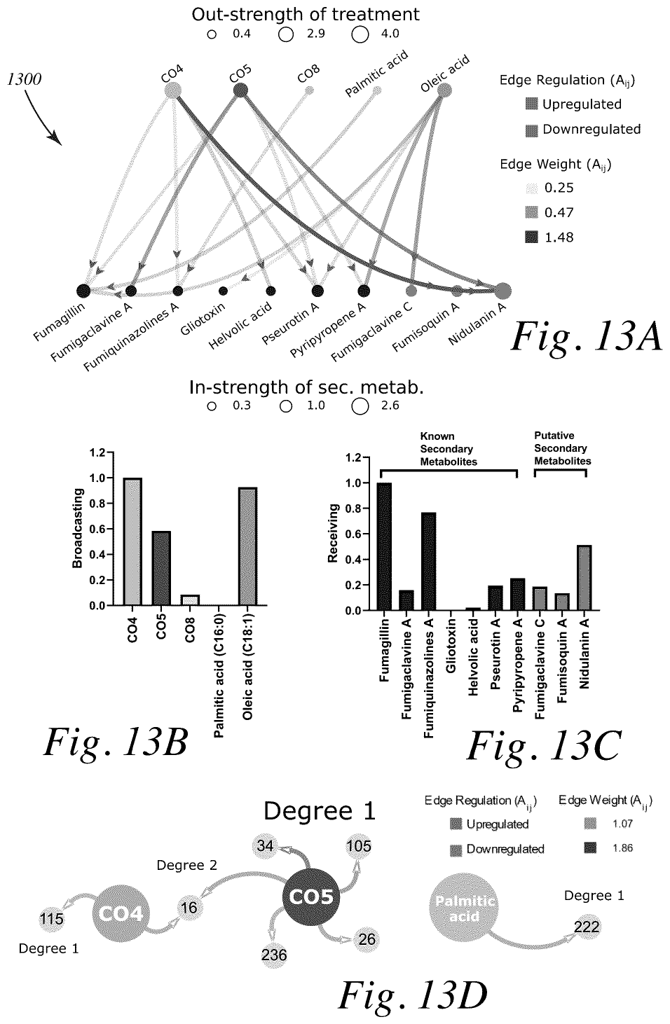

The influence of chitooligosaccharides (i.e., CO4, CO5, and CO8) and lipids (palmitic acid and oleic acid) on the production of known and putative metabolites by Aspergillus fumigatus at 25° C. was analyzed using the direct route as shown in FIGS. 13A-C. The bipartite network 1300 provides a visual representation and enable a clear distinction between the effects of these treatments, as shown in FIG. 13A. While chitooligosaccharides resulted in an up-regulation of the identified metabolites, the lipids showed a down-regulation. The network centrality measure of node out-strength of the treatments reveals that CO4 has the highest effect on triggering metabolite production, followed by CO5, then oleic acid. The treatments CO8 and palmitic acid showed a minor influence on inducing metabolite production. This was expected as those two treatments influence the production of only one metabolite with low values of log₂ fold change. Moreover, the putative metabolite nidulanin A possesses the highest node in-strength amongst the metabolites as it is the most regulated metabolite, influenced by the chitooligosaccharides CO4 and CO5.

The network centrality measure of PageRank considers various factors, such as the number of edges from or to a node and the relative importance of nodes based on their connections to highly and uniquely connected nodes, to determine the most influential nodes in a network. The PageRank measure has been used extensively in various metabolic network analysis. Due to the nature of metabolic interactions, variations in the PageRank measure have also been introduced. For the treatments, the ability to be influential at triggering metabolite production is measured by the broadcasting version of the PageRank measure. In contrast, the ability of metabolites to be receptive to treatments is denoted by the receiving version of the PageRank measure, as shown in FIGS. 13B and 13C, respectively. The results, which are minimum-maximum normalized (values between 0 and 1), indicate that CO4 is the most effective treatment, followed by oleic acid. The bipartite network shows that CO4 triggers six metabolites compared to five triggered by oleic acid. Both treatments trigger two unique metabolites (helvolic acid and fumisoquin A by CO4; gliotoxin and fumigaclavine C by oleic acid). Nonetheless, CO4 has a higher influence on triggering the production of a unique metabolite, fumisoquin A. The broad number of metabolites activated by the CO4 treatment with a more prominent effect shows the wider impact of CO4 on triggering metabolites.

The current modeling framework reveals oleic acid to have a high impact on the production of metabolites even at 25° C. Oleic acid has a higher broadcasting PageRank value than CO5, contrary to the node out-strength values (CO5 has higher out-strength than oleic acid as shown in FIG. 13A). The higher broadcasting PageRank value of oleic acid is attributed to its ability to uniquely trigger two metabolites (gliotoxin and fumigaclavine C) compared to just one by CO5 (fumigaclavine A). Furthermore, palmitic acid has the least broadcasting PageRank measure followed by CO8, contrary to the results of node out-strength (CO8 has the least out-strength as shown in FIG. 13A). This change results from palmitic acid being connected to a highly receptive metabolite, fumagillin, triggered by many treatments. Thus, palmitic acid is not a unique treatment. CO8 is related to a unique metabolite, fumiquinazolines A, which is not triggered by many treatments.

The receiving PageRank measures of the metabolites (FIG. 13C) reveal that the known metabolite fumagillin followed by fumiquinazolines A have much higher receptivity at being triggered by treatments compared to the putative metabolite nidulanin A. This result is contrary to that provided by the node in-strength, which showed nidulanin A to be the most influenced metabolite (FIG. 13A). While nidulanin A is the most regulated metabolite, its production was only activated by CO4 and CO5 treatments, producing many other metabolites. Therefore, the uniqueness of nidulanin A for being triggered is reduced. Furthermore, even though fumiquinazolines A has a much lower node in-strength value than nidulanin A and is only activated by CO4 and CO8, the latter treatment uniquely triggers this metabolite. This unique relationship with fumiquinazolines A is also one of the reasons why CO8 has a higher broadcasting PageRank value than palmitic acid, as discussed above.

These observations shown with the direct route could not be inferred using traditional methods like UpSet or volcano plots. Also, since the gene cluster for nidulanin A has been identified in all Aspergillus spp. and yet it has not been described in A. fumigatus, CO4 and CO5 could be used as treatments for the characterization of this metabolite in A. fumigatus. Lastly, many of these known and putative metabolite peaks might still fall into a peak noise. Although a peak cutoff was initially used in MAVEN to identify bona fide peaks, the auxiliary route can be used to identify known and unknown analytes or metabolites highly produced in response to a particular treatment using an untargeted metabolomics approach.

Auxiliary Route

The auxiliary route follows an untargeted metabolomic profiling of the treatments. The auxiliary route illustrated in FIG. 13D demonstrates the system and method's ability to isolate highly produced known and unknown analytes that exhibit a log₂ fold change greater than 1 or less than −1 for future experimentation and characterization. In the direct route, a peak area cutoff of 5×10⁵ was used to detect signals with significant peaks between a treatment and solvent control. Although their peak area cutoff of 5×10⁵ eliminates most noise, it is a linear cutoff and leaves some residual noise in the analysis due to column creep. Therefore, the dataset for the auxiliary route can be curated from the experimental study using baseline correction preprocessing tools. Following the processes defined by “Dynamics of Metabolite Induction in Fungal Co-cultures by Metabolomics at Both Volatile and Non-volatile Levels”, Front. Microbiol., 5 Feb. 2018 Sec. Antimicrobials, Resistance and Chemotherapy, Azzollini et al., peaks can be picked using GridMass and aligned using RANSAC alignment or another suitable alignment tool. Peak data can then be matched to known profiles in KEGG and LipidMap.

Peak significance was determined upon FCROS scoring. Non-significant analytes were not included in the network. An interactive map of the network illustrating the details of each analyte (m/z ratio, retention times, p-values, etc.) can be generated.

The untargeted extraction of statistically relevant peaks using the auxiliary route can yield a significant number of analytes for potential exploration. The edges and neighbors of the nodes in the network can be used to determine which analytes to be first considered for targeted exploration. Analytes of particular interest express both regulation and control depending on the treatment considered. Additionally, analytes of extreme up- and down-regulation can be of interest along with the node degree values of the analytes. FIG. 13D illustrates significant peaks with log₂ fold change greater than 1 or less than −1. The total number of interactions (edges in the network) found with the auxiliary route is considerably higher compared to the network in the direct route. Thus, the method reveals more information regarding the interactions amongst the treatments and metabolites than the direct route. Interesting artifacts are revealed from the network built using data at 25° C. At 25 C, analytes ID 26 and ID 105 were matched to fraxetin via the KEGG database query. These were found to be upregulated with respect to the CO5 treatment. Analyte ID 222 was matched to ICAS #18 from the LipidMap database. Palmitic acid significantly down regulates ICAS #18 (ID 222) with respect to the control. At the same time, no single highly produced analyte is triggered by all three chitooligosaccharides. Analyte 16, however, is significantly produced by both CO4 and CO5 treatments.

All analytes of log₂ fold change intensity greater than 1 or less than −1 except for analyte ID 16 are of degree 1. When not taking log₂ fold change intensity into account, there exist 41 analytes of degree 1, 25 analytes of degree 2, 8 analytes of degree 3, 3 analytes of degree 4, and 1 analyte of degree 5. Although treatments can commonly start the production of the analytes considered, the treatments used in this example have a higher tendency to uniquely trigger analytes, which agrees with the UpSet and volcano plots and direct route analysis.

The four unknown analytes with log₂ fold change intensity greater than 1 or less than −1 with IDs 16, 34, 115, and 236 are of particular interest. Additional analytes of interest are those with opposing log₂ fold changes between treatments, whereas all remaining analytes within the networks have aligned log₂ fold changes.

### Aspergillus Example @ 37° C.

As another example, the system and method can be utilized to analyze the LC/MS data with treated samples grown at 37° C., which revealed that all individually applied treatments significantly induce the production of analytes compared to the solvent control.

Direct Route

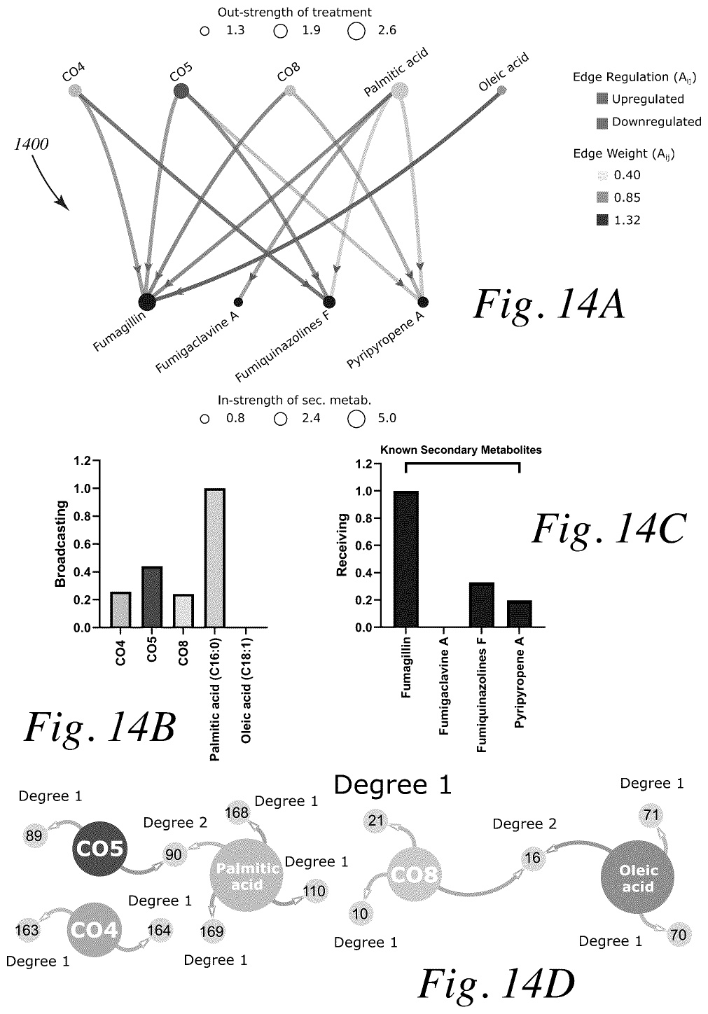

The results of the direct route revealing the influence of the treatments on the production of specialized metabolites in Aspergillus fumigatus at 37° C. are shown in FIGS. 14A-C. As expected, fewer analytes and known or putative specialized metabolites were produced at 37° C. in both solvent controls and treatments, as shown in the bipartite network 1400 of FIG. 14A. Furthermore, there is no clear distinction on how the two treatments regulate the production of metabolites. Both chitooligosaccharides and lipids showed a positive and negative impact on metabolite production at 37° C., whereas, at 25° C., chitooligosaccharides upregulated the production of metabolites and lipids down-regulate it (FIG. 13A). It is worth noting that the metabolites fumagillin and pyripyropene A are the only metabolites that were uniquely triggered at both temperatures; however, the treatments that started the production of these two metabolites were different depending on the temperature. The bipartite network representation further clarifies such pathways and regulations.

Both the node strength and PageRank measures give similar results for identifying the effective treatments and most receptive metabolites, as shown in FIGS. 14B-C. The lipid palmitic acid is the most effective treatment on metabolite production as it resulted in both up- and downregulation of all metabolites analyzed. At 37° C., CO4 and oleic acid are poor at triggering the production of metabolites, contrary to what was observed at 25° C., where these treatments showed the most influence (FIG. 13B). The specialized metabolite fumagillin has the highest receptivity at being triggered by treatments, as shown in FIG. 14C. The same result was observed at 25° C.

Auxiliary Route

Considering the analytes from the network at 37° C. shown in FIG. 14D, twelve significant analyte peaks with log₂ fold change intensities greater than 1 were extracted from the processed data. All three CO treatments at 37° C. triggered no shared analytes. Nor did the two lipid treatments trigger a shared analyte. Ten of the twelve analytes with log₂ fold change intensities greater than 1 or less than −1 are uniquely triggered by a single treatment (i.e., analytes with node degree 1), as shown in FIG. 14D. Of the twelve significant extracted analyte peaks, eleven were identified from database queries. Analytes identified by KEGG were putative metabolites, hellebrigenin 3-acetate (ID 10), fraxetin (ID 16), beta-cyclopiazonate (ID 70), phenylbutazone (ID 71), borrerine (ID 89), clofibrate (ID 110), alangimarine (IDs 163 and 164), and sulindac (IDs 168 and 169). LipidMaps identified 6′-hydroxysiphonaxanthin decenoate (ID 21). The only unidentified analyte was analyte ID 90, upregulated in both CO5 and Palmitic Acid treatments. There are no connections in ID numbers between the 25° C. and 37° C. experiments.

When considering all analytes (not only those with log₂ fold changes greater than 1 or less than −1), there exists five analytes with opposing log₂ fold changes (analytes with IDs 21, 70, 163, 164, and 168) compared to the four analytes at 25 C. Analytes 163 and 168 are both of degree 3. Analyte 163 is upregulated by both palmitic acid and CO8 yet downregulated by CO4. Analyte 168 is upregulated by palmitic acid yet downregulated by both CO4 and oleic acid.

Oleic acid was reported as an inducer of germination in Aspergillus fumigatus at 37° C. None of the known metabolites identified were previously linked to germination in A. fumigatus. Therefore, the system can predict that one of the highly up-regulated unknown analytes may be the culprit behind the increased germination of this fungus at 37° C., which can be the target for future experiments.

These examples of the systems and methods of the present disclosure provide a data-driven modeling framework using network analysis to dissect the connection between exogenous inputs—biological compounds like lipids and chitooligosaccharides—and the metabolomic outputs—putative metabolites and unknown analytes—in the opportunistic human pathogen A. fumigatus. Another example is “Lipo-chitooligosaccharides induce specialized fungal metabolite profiles that modulate bacterial growth.” Msystems 7.6 (2022): e01052-22 by Rush, Tomis A., et al., where they used the same system and methods to show that antibacterial properties and bio-activators were induced by Lipo-chitooligosaccharides (LCOs) when treated with Aspergillus fumigatus. “These findings suggest that LCOs may play an important role in the competitive dynamics of non-plant-symbiotic fungi and bacteria. This study identifies specific metabolomic profiles induced by these ubiquitously produced chemicals and creates a foundation for future studies into the potential roles of LCOs as modulators of interkingdom competition.” (taken from the abstract of the paper).

### Discussion of Applications and Examples

Bipartite networks with two classifications of nodes are built. The network nodes represent the treatments and specialized metabolites under consideration. The edges connecting the nodes represent the magnitude of up- or down-regulation of the specialized metabolites triggered by the corresponding treatments. Two routes to characterize the production of the specialized metabolites are provided: (1) the direct route 1502 (See FIG. 15) for the production of known and putative metabolites and (2) the auxiliary route 1504 (See FIG. 15) for the production of unknown analytes. Moreover, network centrality measures of node strength and PageRank are used to rank the treatments and specialized metabolites 1506. The treatments are ranked based on their ability to trigger the production of various specialized metabolites. The specialized metabolites are ranked based on their ability to be influenced by multiple treatments.

The insights about the most effective treatments and most influenced specialized metabolites are valuable for (1) validating known specialized metabolites through applied exogenous treatments or environmental cues and (2) discovering new specialized metabolites from putative metabolites and unknown analytes by genetic knockouts to characterize their gene clusters as depicted in post-analysis applications 706 (see FIG. 15). Ultimately the system and method facilitate tracking how a treatment will elucidate the production of secondary metabolites. It is widely known that most biosynthetic gene clusters are silent under standard culture conditions resulting in minimal production of secondary metabolites. The present disclosure can help researchers determine how their treatments will improve the production and accumulation of natural products. Those results can be validated through mass spectrometry analysis and comparison to fragmentation patterns from published datasets or commercial standards and through transcriptomic analysis to assess their biosynthetic gene expressions. Further confirmation can be done through knockout experiments and functional validation of the targeted biosynthetic gene clusters in post-analysis applications.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

1. Memory encoding instructions that, when executed by data processing apparatus, cause the data processing apparatus to perform operations comprising:
accessing information relating to effects of chemical treatments on analyte production;
building, based on the accessed information, a bipartite network comprising chemical treatment nodes and analyte nodes, wherein the bipartite network quantitatively represents the effects of chemical treatments to trigger production of analytes;
analyzing the bipartite network to identify dominant chemical treatments among the chemical treatments and identify secondary metabolites among the analytes; and
outputting the identified dominant chemical treatments and the identified secondary metabolites.

2. The memory of claim 0, wherein the analyzing the bipartite network includes at least one of analyzing the bipartite network via a direct route to identify known and putative secondary metabolites and analyzing the bipartite network via an auxiliary route to identify untargeted and unknown analytes of interest.

3. The memory of claim 0, wherein the operations follow a direct route approach such that the analyte nodes of the built bipartite network are either known secondary metabolites or putative secondary metabolites or both, and
wherein the analyzing the bipartite network analysis comprises identifying the most influenced secondary metabolites from among the known or putative secondary metabolites.

4. The memory of claim 3, wherein the building the bipartite network comprises:
defining two bipartite sets of nodes, one of the bipartite sets of nodes including chemical treatments and the other bipartite sets of nodes including analytes;
constructing directional, weighted edges between nodes using log2fold change of an analyte by a chemical treatment; and
assigning positive or negative sign to each edge for visualization of metabolite upregulation or metabolite downregulation.

5. The memory of claim 3, wherein the analyzing the bipartite network analysis comprises:
computing a plurality of network centrality measures of the bipartite network including:
out-degrees for each chemical treatment;
in-degrees for each analyte;
broadcasting rank for each chemical treatment; and
receiving rank for each analyte.

6. The memory of anyone of claim 5, wherein the broadcasting ranks and receiving ranks are normalized PageRank measures.

7. The memory of claim 1, wherein the operations follow an auxiliary route approach, and wherein the analyzing the bipartite network includes analyzing the bipartite network to identify untargeted and unknown analytes of interest.

8. The memory of claim 1, wherein the information relating to effects of chemical treatments on analyte production comprises liquid chromatography mass-spectroscopy (LCMS) spectra of the analytes corresponding to the chemical treatments.

9. Memory encoding instructions that, when executed by data processing apparatus, cause the data processing apparatus to perform operations comprising:
accessing spectra of unknown analytes relating to chemical treatments;
generating a matrix relating the spectra of the unknown analytes to the chemical treatments;
applying fold change rank order statistics (FCROS) to the matrix to determine a p-value and an f-value for each unknown analyte;
building a bipartite network using unknown analytes with statistically significant p-values and f-values;
selecting one or more unknown analytes by fold change or edge degree; and
identifying secondary metabolites from among the selected one or more unknown analytes.

10. The memory of claim 9, wherein the applying fold change rank order statistics (FCROS) to the matrix comprises:
repeatedly, for all combinations of controls and treatments:
selecting a control sample and a treatment sample;
computing a fold change for each analyte;
ranking analytes in increasing order to obtain an associated rank with each analyte;
computing an average of ranks for each analyte;
using the mean and variance of the average of ranks to generate a normal distribution to associate a probability with each rank; and
defining two cutoff values to identify up- and down-regulated analytes, wherein an analyte is downregulated if below a first cutoff value and an analyte is upregulated if above a second cutoff value.

11. The memory of claim 9, wherein the building the bipartite network comprises:
repeatedly, for each treatment:
selecting a treatment-specific FCROS matrix;
in response to an analyte in the matrix having significant f-value and p-value, generating a treatment graph connecting all analyte nodes to a single node representing a treatment type associated with the treatment-specific FCROS matrix;
represent edges between nodes and treatment type by fold change;
unioning the treatment graphs to generate a full union of all graphs and a network of similar treatments.

12. The memory of claim 9, wherein the selecting one or more unknown analytes by fold change or edge degree includes scoring the one or more analytes by at least one of:
degree connected to a singular treatment;
upregulation value;
downregulation value; and
shared analytes between similar treatments.

13. The memory of claim 9, wherein the selecting one or more unknown analytes by degrees indicative of production of an unknown analyte.

14. The memory of claim 9, wherein the spectra of unknown analytes relating to chemical treatments comprises liquid chromatography mass-spectroscopy (LCMS) spectra of the analytes corresponding to the chemical treatments.

15. A method for recommending usage of chemical treatments, the method comprising:
accessing information relating to effects of chemical treatments on analyte production;
building, based on the accessed information, a bipartite network comprising chemical treatment nodes and analyte nodes, wherein the bipartite network quantitatively represents the effects of chemical treatments to trigger production of analytes;
analyzing the bipartite network to identify dominant chemical treatments among the chemical treatments and identify secondary metabolites among the analytes; and
outputting the identified dominant chemical treatments and the identified secondary metabolites.

16. The method of claim 15, wherein the analyzing the bipartite network includes at least one of analyzing the bipartite network via a direct route to identify known and putative secondary metabolites and analyzing the bipartite network via an auxiliary route to identify untargeted and unknown analytes of interest.

17. The method of claim 15, wherein the analyzing follows a direct route approach such that the analyte nodes of the built bipartite network are either known secondary metabolites or putative secondary metabolites or both, and
wherein the analyzing the bipartite network analysis comprises identifying the most influenced secondary metabolites from among the known or putative secondary metabolites.

18. The method of claim 17, wherein the building the bipartite network comprises:
defining two bipartite sets of nodes, one of the bipartite sets of nodes including chemical treatments and the other bipartite sets of nodes including analytes;
constructing directional, weighted edges between nodes using log2fold change of an analyte by a chemical treatment; and
assigning positive or negative sign to each edge for visualization of metabolite upregulation or metabolite downregulation.

19. The memory of claim 15, wherein the analyzing the bipartite network analysis comprises:
computing a plurality of network centrality measures of the bipartite network including:
out-degrees for each chemical treatments;
in-degrees for each analyte;
broadcasting rank for each chemical treatment; and
receiving rank for each analyte.

20. The method of claim 15, wherein the analyzing the bipartite network includes analyzing the bipartite network via an auxiliary route to identify untargeted and unknown analytes of interest.

21. The method of claim 20 wherein analyzing the bipartite network includes:
accessing spectra of unknown analytes relating to chemical treatments;
generating a matrix relating the spectra of the unknown analytes to the chemical treatments;
applying fold change rank order statistics (FCROS) to the matrix to determine a p-value and an f-value for each unknown analyte;
building a bipartite network using unknown analytes with statistically significant p-values and f-values;
selecting one or more unknown analytes by fold change or edge degree; and
identifying secondary metabolites from among the selected one or more unknown analytes.
